(19) United States
(12) Patent Application Publication
(10) Pub. No.: US 2025/0037838 A1
(43) Pub. Date: Jan. 30, 2025

(54) SYSTEMS AND METHODS FOR INDUCING WELLNESS IN A USER

(71) Applicant: REFLECT INNOVATION LTD., Herzliya (IL)

(72) Inventors: Nir KLEIN, El-Rom (IL); Noga SAPIR, Tel Aviv (IL)

(21) Appl. No.: 18/715,892

(22) PCT Filed: Dec. 12, 2022
(86) PCT No.: PCT/IL2022/051310

§ 371 Date: Jun. 3, 2024

Related U.S. Application Data
(60) Provisional application No. 63/290,118, filed on Dec. 16, 2021.

(51) Int. Cl. G16H 20/70; G16H 40/40

(52) U.S. Cl. CPC G16H 20/70; G16H 40/40

(57) ABSTRACT

A wellness inducing system, including an apparatus configured to be proximal to a user, the apparatus including at least one sensor configured to collect signals associated with a current session of a user, and an indicator having at least two modes of operation, a processor in communication with a memory module, the memory module having stored thereon program code, the program code executable by the processor to: receive at least one signal from the at least one sensor and apply, to the apparatus, a set of instructions for the current session, wherein the set of instructions includes operational parameters for the at least two modes of operation, and wherein the set of instructions is based, at least in part, on output of an algorithm configured to: receive raw and/or processed data associated with at least one previous session of the user, apply the received data to a plurality of configurations, score each of the plurality of configurations, thereby obtaining a plurality of scores, each of the scores being values associated with key performance indicator(s) (KPI) of the user, identify at least one of the plurality of configurations as having a preferred score (or KPI value), and generate the set of instructions for the current session, based, at least in part, on the at least one of the plurality of configurations.

### TECHNICAL FIELD

The present disclosure relates generally to systems and methods for inducing wellness in a subject, and, more specifically, systems and methods implementing an adaptive algorithm for inducing wellness in a subject.

### BACKGROUND

Medical research has shown that stress has an effect on health that can be harmful to the body. Stress, by definition, raises a person's blood pressure. Hypertension can increase the risk of a heart attack or stroke. Stress raises heart rate, blood pressure, blood glucose levels, and increased activity of the sympathetic nervous system and may result in a fight-or-flight response which is a physiological change in order to prepare the body for physical activity. Further, relaxed people can make better decisions and learn better.

There are many causes for stress, such as environmental factors such as living under overcrowded and increasingly noisy conditions, experiencing daily events such as coping with traffic, physical activity, undergoing major life changes such as a divorce or death of a loved one, stress in the workplace and at home, and finally the use of chemical agents such as alcohol or drugs as a consequence of these stressors. Stress response can be measured by measuring parameters, such as heart rate, blood pressure, skin conductivity and the like. There is thus a need for an apparatus, a system and a method for reducing stress and/or inducing mindfulness

### SUMMARY

Aspects of the disclosure, according to some embodiments thereof, relate to systems and methods of inducing wellness in a subject.

According to some embodiments there is provided an algorithm that provides adaptive biofeedback based, at least in part on, previous data. According to some embodiments, as more data is collected from previous sessions, the biofeedback may be more personalized and accurate for a specific subject. According to some embodiments, the algorithm is configured remotely prior to the next sessions. According to some embodiments, the algorithm may be configured to provides adaptive biofeedback based, at least in part on, metadata associated with the subject. According to some embodiments, the algorithm may be configured to analyze the metadata associated with the subject.

According to some embodiments there is provided an algorithm that provides adaptive biofeedback based, at least in part on, data received from two or more sensors. In particular, the algorithm may provide the adaptive biofeedback based, at least in part on data received from an accelerometer. According to some embodiments, the algorithm may provide the adaptive biofeedback based, at least in part on data received from an ECG sensor. According to some embodiments, the algorithm may provide the adaptive biofeedback based, at least in part on an analysis of the ECG data using the accelerometer data, thereby interpreting ECG fluctuations caused by movement of the subject. According to some embodiments, the algorithm may be configured to interpret and/or modify the ECG signal using data received from the accelerometer.

Advantageously, analyzing data received from the ECG and the accelerometer sensors together may enable the algorithm to eliminate noise from the signal by matching it with movement data.

According to some embodiments there is provided an algorithm that provides adaptive biofeedback through an apparatus for inducing wellness in a subject. According to some embodiments, the algorithm that provides adaptive biofeedback by operating one or more indicators of the apparatus. According to some embodiments, the one or more indicators may include two or more modes of operation, wherein the operation of the indicators may include changing between the two or more modes at a specified rate, frequency, intensity, and the like. According to some embodiments, the modes of operation of the indicators may include auditory, vibration, visual, tactile, temperature, shape and/or applied pressure changes.

Advantageously, since each individual subject has their own physiological characteristics (e.g., hands sizes, skin types, grip, environment, skin thermal and electrical conductivity), the algorithm that provides adaptive biofeedback enables each individual subject to receive a tailored experience while using the apparatus for inducing wellness.

According to some embodiments there is provided an algorithm configured to receive a set of instructions for a current session of a subject. According to some embodiments, the set of instruction s may include data associated with operation of the indicators of the apparatus. According to some embodiments, and as explained in greater detail elsewhere herein, the set of instruction may be based on one or more configurations.

According to some embodiments, there is provided a method for producing a set of instructions for a current session of a subject. According to some embodiments, the method may include gathering data during a previous session of the subject and/or receiving data associated with a previous session of the subject. According to some embodiments, the method may include analyzing the data. According to some embodiments, the method may include running the data through one or more optional configurations, and receiving an output associated with an outcome of the data within the configuration. According to some embodiments, the method may include choosing at least one configuration for which the outcome was most desirable. According to some embodiments, the method may include sending, to the apparatus, a set of instructions for a current session of the subject, based, at least in part, on the at least one chosen configuration.

According to some embodiments there is provided a wellness inducing system, including: an apparatus configured to be proximal to a user, the apparatus including: at least one sensor configured to collect signals associated with a current session of a user, and an indicator having at least two modes of operation, a processor in communication with a memory module, the memory module having stored thereon program code, the program code executable by the processor to: receive at least one signal from the at least one sensor, and apply, to the apparatus, a set of instructions for the current session, wherein the set of instructions includes operational parameters for the at least two modes of operation, and wherein the set of instructions is based, at least in part, on output of an algorithm configured to: receive raw and/or processed data associated with at least one previous session of the user, apply the received data to a plurality of configurations, score each of the plurality of configurations, thereby obtaining a plurality of scores, each of the scores being values associated with key performance indicator(s) (KPI) of the user, identify at least one of the plurality of configurations as having a preferred score (or KPI value), and generate the set of instructions for the current session, based, at least in part, on the at least one of the plurality of configurations.

According to some embodiments, there is provided a method for inducing wellness, including, receiving at least one signal associated with a current session of a user, from at least one sensor positioned on an apparatus configured to be proximal to the user, applying to the apparatus, a set of instructions for a current session, wherein the set of instructions includes operational parameters for at least two modes of operation of an indicator of the apparatus, wherein the set of instructions is based, at least in part, on output of an algorithm configured to: receive raw and/or processed data associated with at least one previous session, apply the received data to a plurality of configurations, score each of the plurality of configurations, thereby obtaining a plurality of scores, each of the scores being values associated with key performance indicator(s) (KPI) of the user, identify at least one of the plurality of configurations as having a preferred score (or KPI value), and generate the set of instructions for the current session, based, at least in part, on the at least one of the plurality of configurations.

According to some embodiments, there is provided a wellness inducing system, including: an apparatus configured to be proximal to a user, the apparatus including: at least one sensor configured to collect signals associated with a current session of a user, and an indicator having at least two modes of operation, a processor in communication with a memory module, the memory module having stored thereon program code, the program code executable by the processor to: receive at least one signal from the at least one sensor, apply, to the apparatus, a set of instructions for the current session, wherein the set of instructions includes operational parameters for the at least two modes of operation, and wherein the set of instructions is based, at least in part, on output of an algorithm configured to: receive raw and/or processed data associated with the at least one signal in real time, apply the received data to a plurality of configurations, score each of the plurality of configurations, thereby obtaining a plurality of scores, each of the scores being values associated with key performance indicator(s) (KPI) of the user, identify at least one of the plurality of configurations as having a preferred score (or KPI value), and generate the set of instructions for the current session, based, at least in part, on the at least one of the plurality of configurations.

According to some embodiments there is provided a method for inducing wellness, including, receiving at least one signal associated with a current session of a user, from at least one sensor positioned on an apparatus configured to be approximal to the user, applying to the apparatus, a set of instructions for a current session, wherein the set of instructions includes operational parameters for at least two modes of operation of an indicator of the apparatus, wherein the set of instructions is based, at least in part, on output of an algorithm configured to: receive raw and/or processed data associated with the at least one signal in real time, apply the received data to a plurality of configurations, score each of the plurality of configurations, thereby obtaining a plurality of scores, each of the scores being values associated with key performance indicator(s) (KPI) of the user, identify at least one of the plurality of configurations as having a preferred score (or KPI value), and generate the set of instructions for the current session, based, at least in part, on the at least one of the plurality of configurations.

According to some embodiments, a higher value of the score is indicative of a better KPI. According to some embodiments, the signals associated with a current session of a user include at least one of a signal associated with a wellness state of the user and/or a signal associated with raw data of the current session. According to some embodiments, signals associated with the wellness state of the user include physiological measurements from which the wellness state of the user can be deduced.

According to some embodiments, the raw data includes environmental factors of the surrounding which can affect physiological measurements of the user. According to some embodiments, the raw data includes user specific data, the user specific data including any one or more of the age, gender, medical history, user reported data, and data that is passively collected from the user. According to some embodiments, the processed data includes data associated with the wellness state of the user based, at least in part, on analyzed raw data (of the current session and/or of the previous session).

According to some embodiments, the set of instructions includes optional operational parameters for the at least two modes of operation of the indicator, to be implemented based on (or in correlation to) the signals associated with a current session of a user. According to some embodiments, each configuration depicts one or more optional sets of instructions. According to some embodiments, the program code is further executable to identify the user. According to some embodiments, the at least two modes of operation are configured to sync with signals associated with a current session of the user.

According to some embodiments, the signals are associated with at least one of a breathing rate, heart rate, and movement of the user. According to some embodiments, the at least two modes of operation are configured to sync to a rate being lower than a breathing rate or heart rate and movement of the user, by about 5-30%. According to some embodiments, the at least one of previous sessions includes a plurality of previous sessions. According to some embodiments, the program code is further executable to store and/or record data associated with the user, the data including any one or more of signals obtained during the session, one or more configurations having one or more scores indicating the KPI of the user, environmental data, data inputted by the user, and the set of instructions of the session.

According to some embodiments, the algorithm is further configured to implement one or more template configurations for the plurality of configurations. According to some embodiments, the algorithm is further configured to change at least one of the one or more template configurations based, at least in part, on data of the at least one previous session of the user. According to some embodiments, generating the set of instructions for the current session is based, at least in part, on a subset of the plurality of configuration, wherein the scores of the subset of the plurality of configurations are associated with desired KPI values.

According to some embodiments, generating the set of instructions for the current session is based, at least in part, on at least two configurations, wherein each of the at least two configurations is associated with data of different previous sessions of the user. According to some embodiments, the algorithm is further configured to generate a scoring scale configured to score the KPI of at least one of the plurality of configurations with the received data based, at least in part, on data of the at least one previous session of the user.

According to some embodiments, the algorithm is further configured to generate a typecast of the user, and wherein the scoring scale is calculated using the typecast of the user. According to some embodiments, the typecast includes one or more identifying features of the user. According to some embodiments, the algorithm is further configured to generate a typecast of the user based on signals associated with the current session.

According to some embodiments, the algorithm is further configured to generate the set of instructions for the current session based, at least in part, on any one or more of the age, gender, environmental factors of the current session and/or geographical region, user inputted data, raw data of the user and/or the current session, processed data associated with the user and/or with the current session, medical history of the user, user reported data, and data that is passively collected from the user.

According to some embodiments, the at least one sensor includes one or more sensors includes galvanic skin response (GSR), electrocardiogram (ECG), photoplethysmogram (PPG), thermometer, accelerometer, barometer, eye tracker, GPS, electromyography sensor (EMG), pupil size detector, pupil location and/or movement tracker, oximeter, pressure sensor, blood pressure sensor, gyroscope, microphone, camera, fingerprint sensor, strain gauge proximity sensor, fingerprint sensor, (humidity sensor, optical sensor, light sensor, photodiode, photoresistor, audio sensor, or any combination thereof.

According to some embodiments, the at least one sensor includes at least one accelerometer and at least one ECG, and wherein the processed data includes ECG signals that are analyzed and/or modified using signals of the accelerometer, thereby eliminating noise of the ECG signals. According to some embodiments, the apparatus includes at least one pair of indentations on an outer surface thereof, configured for placing one or more fingers from at least one hand of the user, and wherein at least one sensor is positioned around or within each of the indentations such that the sensor is configured to receive signals associated with a physiological state of the user.

According to some embodiments, the indicator is configured to provide visual, audio, tactile feedback information or any combination thereof. According to some embodiments, the indicators include a light source. According to some embodiments, the operational parameters of a first mode of operation and a second mode of operation include any one of different light wavelengths, light intensities, durations, rhythms, frequencies, pulses, cycles, iterations, or any combination thereof.

According to some embodiments, the indicator further includes a mechanism for changing the shape and/or size of the apparatus. According to some embodiments, the operational parameters of a first mode of operation and a second mode of operation include any one of a different shape and/or size of the apparatus, different rhythms, frequencies, and/or pulses of change of the shape and/or size of the apparatus, or any combination thereof.

According to some embodiments, the indicator further includes a temperature changing device configured to change the temperature of at least a portion of an outer surface of the apparatus. According to some embodiments, the operational parameters of a first mode of operation and a second mode of operation include any one of different temperatures of at least a portion of the surface of the apparatus, different rhythms, frequencies, and/or pulses of change in temperatures, or any combination thereof.

According to some embodiments, the indicator further includes a device configured to output audio. According to some embodiments, the operational parameters of a first mode of operation and a second mode of operation include any one of different audio outputs, rhythms, frequencies, pulses, wavelengths, and intensities of the audio outputs, or any combination thereof. According to some embodiments, the indicator further includes a device configured to generate vibrations.

According to some embodiments, the operational parameters of a first mode of operation and a second mode of operation include any one of different vibration outputs, rhythms, frequencies, pulses, wavelengths, and intensities, or any combination thereof. According to some embodiments, the at least one signal is associated with any one or more of the heart rate variability (HRV), the heart rate (HR), the respiratory rate (RR), temperature, movements, and movement patterns of the user, or any combination thereof.

According to some embodiments there is provided a biofeedback method of inducing wellness including: positioning the apparatus in proximity to a user, and receiving at least two indications from the indicator, thereby self-adjusting the wellness level of the user. According to some embodiments, positioning the apparatus in proximity to the user includes positioning the apparatus in contact with the user.

According to some embodiments, the program code is further executable to identify the user based, at least in part, on the at least one signal. According to some embodiments, the algorithm is further configured to receive raw and/or processed data associated with at least one previous session.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In block diagrams and flowcharts, optional elements/components and optional stages may be included within dashed boxes.

In the figures:

FIG. 1 shows a wellness inducing system, in accordance with some embodiments of the present invention;

FIG. 2 shows a block diagram of a wellness inducing system, in accordance with some embodiments of the present invention;

FIG. 3 shows a schematic illustration of a program code for inducting wellness in a subject, in accordance with some embodiments of the present invention;

FIG. 4 shows a flow diagram of steps of a method for generating a set of instructions, in accordance with some embodiments of the present invention; and

FIG. 5 shows a flow diagram of steps of a method for generating a set of instructions in real time, in accordance with some embodiments of the present invention.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the following description, various aspects of the invention will be described. For the purpose of explanation, specific details are set forth in order to provide a thorough understanding of the invention. However, it will also be apparent to one skilled in the art that the invention may be practiced without specific details being presented herein. Furthermore, well-known features may be omitted or simplified in order not to obscure the invention.

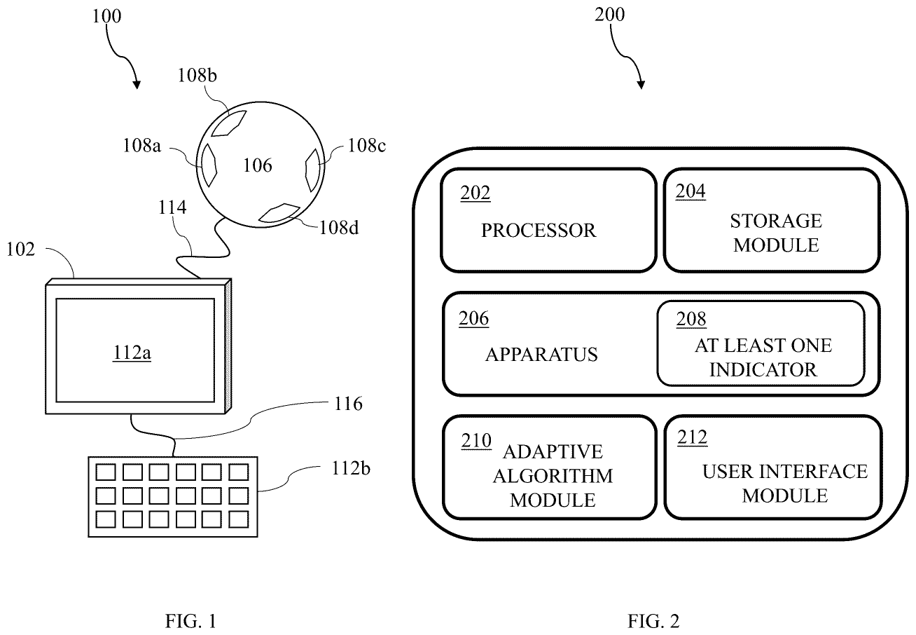

Throughout the following description, similar elements of different embodiments of the system are referenced by element numbers differing by integer multiples of 100. For example, an apparatus of FIG. 1 is referenced by the number 106, and an apparatus of FIG. 2, which corresponds to apparatus 106 of FIG. 1, is referenced by the number 206.

Reference is made to FIG. 1, which shows a wellness inducing system, in accordance with some embodiments of the present invention.

According to some embodiments, the system 100 is configured for inducing wellness in a subject. According to some embodiments, the system 100 may include an apparatus 106 configured to be proximal to a subject. According to some embodiments, the system 100 may include a processor 102 configured to be in communication with the apparatus 106. According to some embodiments, the processor 102 may be an integral part (or in other words, within the same unit as) the apparatus 106. According to some embodiments, the system 100 may include a subject interface module 112 configured to enable a subject to interact with the processor 106 and in communication with the processor 106. According to some embodiments, the system 100 and/or the apparatus 106 may include one or more indicators 108 configured to interact with the subject (or user). According to some embodiments, the processor 102 may be in operable communication with the one or more indicators 108 such that the one or more indicators 108 may receive commands from the processor 102.

According to some embodiments, the apparatus 106 may include a body configured to be held by the subject. According to some embodiments, the apparatus 106 may be configured to be positioned proximal and/or abutting to the subject. According to some embodiments, the apparatus 106 may include a substantially spherically shaped body. According to some embodiments, the apparatus 106 may include one or more indentations and/or pairs of indentations located on an outer surface of the body. According to some embodiments, the apparatus may include one or more pairs of indentations on an outer surface of the apparatus body, which may be configured for placing one or more fingers from at least one hand of the subject.

According to some embodiments, the apparatus 106 may include a first and the second pair of indentations, which may be located on opposing sides of the body surface. According to some embodiments, the indentations may be suitable for placing corresponding fingers from each hand of the subject.

According to some embodiments, the apparatus may include one or more sensors positioned therein. According to some embodiments, the apparatus may include one or more sensors positioned around or within one or more of the indentations, such that the sensor is configured to receive signals associated with a physiological state of the subject. According to some embodiments, the one or more sensors may be in communication with the processor 102.

According to some embodiments, the apparatus may include one or more sensors, configured to detect one or more parameters during usage of the apparatus by a subject (such as, e.g., during a session of usage of the apparatus). According to some embodiments, the one or more sensors may be configured to collect signals associated with a current session of a subject. According to some embodiments, the one or more parameters may include any one or more of a breathing rate, heart rate, and movement of the subject. According to some embodiments, the one or more sensors may be configured to receive one or more signals associated with any one or more of the heart rate variability (HRV), the heart rate (HR), the respiratory rate (RR), temperature, movements, and movement patterns of the user/subject, or any combination thereof. According to some embodiments, the one or more parameters may include any one or more of the signals received from the one or more sensors.

According to some embodiments, the one or more sensors of the apparatus may be detachable. According to some embodiments, the one or more sensors of the apparatus may be separated from the apparatus. According to some embodiments, the one or more sensors of the apparatus may be sensors belonging to a separate device (i.e., a separate monitor, a smartphone and/or a smartwatch) configured to collect data from the user.

According to some embodiments, and as described in greater detail elsewhere herein, the one or more parameters may be wherein the one or more sensors are configured to sense one or more parameters associated with the subject's stress and/or mindfulness state. According to some embodiments, the sensors may include one or more sensors includes galvanic skin response (GSR), electrocardiogram (ECG), photoplethysmogram (PPG), thermometer, accelerometer, barometer, eye tracker, GPS, electromyography sensor (EMG), pupil size detector, pupil location and/or movement tracker, oximeter, pressure sensor, blood pressure sensor, gyroscope, microphone, camera, fingerprint sensor, strain gauge proximity sensor, fingerprint sensor, (humidity sensor, optical sensor, light sensor, photodiode, photoresistor, audio sensor, or any combination thereof. According to some embodiments, the apparatus may include two or more sensors, which may include one or more accelerometers and one or more ECG sensors, configured (and/or positioned in relation to each other) such that the ECG signals that can be analyzed and/or modified using signals of the accelerometer, thereby eliminating noise within the data and/or signals of the ECG.
and an indicator having at least two modes of operation, a first mode being indicative of a first stress and/or mindfulness state of the subject and a second mode being indicative of a second stress and/or mindfulness state of the subject.

According to some embodiments, the one or more indicator 108a/108b/108c/108d (collectively referred to herein as one or more indicators 108) may be in communication with the processor 102. According to some embodiments, the one or more indicators 108 may be positioned within and/or on the apparatus 106. According to some embodiments, the one or more indicators 108 may be positioned along at least a portion of an outer surface of the apparatus 106. According to some embodiments, the one or more indicators 108 may be detachable from the apparatus 106. According to some embodiments, the indicator 108 may be configured to provide visual, audio, tactile feedback information or any combination thereof. According to some embodiments, the one or more indicators 108 may have at least two modes of operation. According to some embodiments, each of the at least two modes of operation may include two or more modes in which the operational parameters of the indicators 108 may be different (in relation to each other).

According to some embodiments, the one or more indicators 108 may include one or more light sources. According to some embodiments, the one or more light sources may be configured to generate light at one or more different wavelengths and/or intensities. According to some embodiments, the two or more operational parameters of the indicators 108 may include any one of different light wavelengths, light intensities, durations, rhythms, frequencies, pulses, cycles, iterations, or any combination thereof.

For example, the two modes of operation may include an “off” mode in which the indicator is not generating light and an “on” mode in which the indicator is generating a light. For example, the two modes of operation may include a first mode in which the wavelength of the light generated by the indicator 108 is a first color (or ranges between a first range of wavelengths) and within the second mode the wavelength of the light generated by the indicator 108 is a second color that is different than the wavelengths of light of the first color (or, alternatively, a second range of wavelengths which ranges at different wavelengths than the first range of wavelengths).

According to some embodiments, the light of the indicator 108 may change during a session. According to some embodiments, the light may change in a cycle. According to some embodiments, the two or more modes may be changing at a specific frequency or at different iterations. According to some embodiments, the two or more modes of the indicator 108 may change between minimal and maximal values, at a specific rate. According to some embodiments, the minimal and/or maximal values may change during the session. According to some embodiments, the minimal and/or maximal values may change during the session in correlation with the breathing rate of the subject. According to some embodiments, the minimal and/or maximal values may change during the session in correlation with the heart rate of the subject. According to some embodiments, the minimal and/or maximal values may change during the session in correlation with the movement type and/or frequency of the subject. According to some embodiments, the minimal and/or maximal values may change such that the light is brighter or lower, for example. According to some embodiments, the minimal and/or maximal values may change such that the light changes in intensity at a higher or lower rate within the session. For example, for a user who has a breathing rate of 10 breathes per minute, the frequency of the light cycle may be 8 cycles per minute.

According to some embodiments, the one or more indicators 108 may include one or more mechanisms for changing the shape and/or size of the apparatus 106. According to some embodiments, the mechanism for changing the shape and/or size of the apparatus 106 may include an inflatable and/or deflectable balloon. According to some embodiments, the mechanism for changing the shape and/or size of the apparatus 106 may include an inflation and/or deflation mechanism. According to some embodiments, the mechanism for changing the shape and/or size of the apparatus may include an actuator (or the like) positioned therein and configured to push against an outer surface of the apparatus 106. According to some embodiments, the outer surface of the apparatus 106 may be compliant to the actuator 108. According to some embodiments, the operational parameters of the two or more modes of operation may include any one or more of a different shapes and/or sizes of the apparatus 106, different rhythms, frequencies, and/or pulses of change of the shapes and/or sizes of the apparatus 106, or any combination thereof.

According to some embodiments, the one or more indicators 108 may include a temperature changing device configured to change the temperature of at least a portion of an outer surface of the apparatus 106. According to some embodiments, the temperature changing device may be positioned near and/or on a portion of an outer surface of the apparatus 106. According to some embodiments, the operational parameters of the two or more modes of operation may include any one or more of different temperatures of at least a portion of the surface of the apparatus, different rhythms, frequencies, and/or pulses of change in temperatures, or any combination thereof.

According to some embodiments, the one or more indicators 108 may include a device configured to output audio. According to some embodiments, the operational parameters of the two or more modes of operation may include any one or more of different audio outputs, rhythms, frequencies, pulses, wavelengths, and intensities of the audio outputs, or any combination thereof.

According to some embodiments, the one or more indicators 108 may include a device configured to generate vibrations. According to some embodiments, the device may be positioned within the apparatus 106. According to some embodiments, the device may be positioned near and/or on at least a portion of an outer surface of the apparatus. According to some embodiments, the device may be positioned such that during operation thereof, a subject holding the apparatus 106 may feel the vibrations of the device (and/or the vibration of the apparatus 106 due to vibrations of the device). According to some embodiments, the operational parameters of the two or more modes of operation may include any one or more of different vibration outputs, rhythms, frequencies, pulses, wavelengths, and intensities, of the vibrations of the device, or any combination thereof.

According to some embodiments, the one or more indicators 108 may receive commands from the processor 102. According to some embodiments, the apparatus 106 may be in communication with the processor 102. According to some embodiments, the processor 102 may be positioned within the apparatus 106 and/or an integral part of the apparatus 106. According to some embodiments, the processor may be separate from the apparatus and coupled thereto using one or more connectors 114.

According to some embodiments, the processor 102 may be in communication with the subject interface module 112a/112b (collectively referred to herein as subject interface module 112). According to some embodiments, the processor 102 may be in communication with the subject interface module 112 via one or more connectors 116. According to some embodiments, the subject interface module may include any one or more of a display (such as, for example, display 112a), a keyboard (such as, for example, keyboard 112b), one or more buttons, a remote control, and the like. According to some embodiments, the connectors 114/116 may be wired or wireless. According to some embodiments, the connectors 114/116 may be any one or more of a cable, Wi-Fi connection, Bluetooth connection, Bluetooth Low Energy connection, and the like.

According to some embodiments, the display 112a may be separate from the apparatus. According to some embodiments, the display may be a display of a separate device, such as, for example, a smartphone and/or smartwatch. According to some embodiments, the display may be configured to output data and/or display data associated with the session of the user.

Reference is made to FIG. 2, which shows a block diagram of a wellness inducing system, in accordance with some embodiments of the present invention.

According to some embodiments, the system 200/100 may include a processor 202/102 in communication with a storage module 204. According to some embodiments, the storage module 204 may have stored thereon one or more program codes configured to operate one or more portions of the apparatus 206/106, such as, for example, the one or more indicators 208/108 and/or the one or more sensors. According to some embodiments, the processor 202/102 may be in communication with the subject interface module 212/112.

According to some embodiments, the system 200/100 may include an adaptive algorithm module 210. According to some embodiments, the adaptive algorithm module 210 may be stored within the storage module 204. According to some embodiments, the program code stored within the storage module 204 may be derived from the adaptive algorithm module 210.

According to some embodiments, the program code may be executable by the processor 202/102 to induce wellness in a subject. According to some embodiments, the program code may be executable by the processor 202/102 to receive at least one signal from the at least one sensor. According to some embodiments, the program code may be executable by the processor 202/102 to apply, to the apparatus, a set of instructions for the current session of the subject. According to some embodiments, and as described in greater detail elsewhere herein, the set of instructions may include operational parameters for the two or more modes of operation of the one or more indicators 208/108 of the apparatus 206/106.

According to some embodiments, the set of instructions may be based, at least in part, on output of an adaptive algorithm, such as the algorithm stored within the adaptive algorithm module 210. According to some embodiments, the algorithm may be configured to receive raw and/or processed data associated with at least one previous session of the subject. According to some embodiments, the algorithm may be configured to receive one or more signals from the one or more sensors of the apparatus 206/106. According to some embodiments, the algorithm may be configured to receive data associated with one or more signals from the one or more sensors of the apparatus 206/106. According to some embodiments, the algorithm may be configured to apply the received data to a plurality of configurations. According to some embodiments, the algorithm may be configured to score each of the plurality of configurations, thereby obtaining a plurality of scores, each of the scores being values associated with key performance indicator(s) (KPI) of the subject. According to some embodiments, the algorithm may be configured to identify at least one of the plurality of configurations as having a preferred score (or KPI value). According to some embodiments, the algorithm may be configured to generate the set of instructions for the current session, based, at least in part, on the at least one of the plurality of configurations.

According to some embodiments, the program code and/or the algorithm may include one or more steps of the methods 400/500 as described hereinbelow.

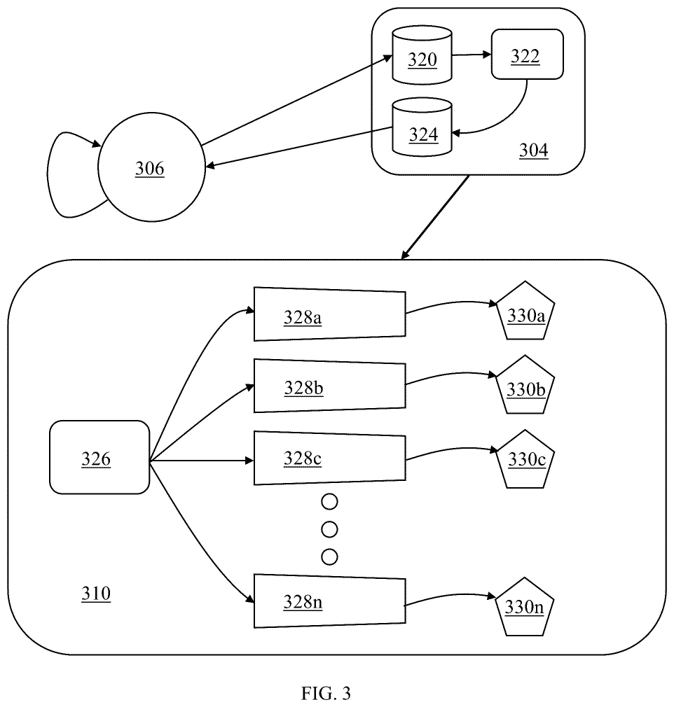

Reference is made to FIG. 3, which shows a schematic illustration of a program code for inducting wellness in a subject, in accordance with some embodiments of the present invention.

According to some embodiments, the subject using the apparatus 306/206/106 may include that the apparatus 306/206/106 performs the current session using a set of instructions. According to some embodiments, the set of instructions of the current session may be received by the processor 202/102 prior to the beginning of the current session. According to some embodiments, the set of instructions of the current session may be generated by the processor 202/102 prior to the beginning of the current session. According to some embodiments, the set of instructions of the current session may be received by the processor 202/102 during the current session. According to some embodiments, the set of instructions of the current session may be generated by the processor 202/102 during the current session.

According to some embodiments, the subject may use the apparatus 306/206/106 during a session (such as, for example, the current session). According to some embodiments, the system 200/100 may be configured to monitor and/or record the session of the subject. According to some embodiments, the system 200/100 may be configured to monitor and/or record the session of the subject by monitoring and/or recording the signals of the one or more sensors of the apparatus 306/206/106. According to some embodiments, the system 200/100 may be configured to monitor and/or record the session of the subject by monitoring and/or recording data received from the one or more sensors of the apparatus 306/206/106.

According to some embodiments, the signals, or in other words, the signals associated with the current session, may include a signal associated with a wellness state of the user and/or a signal associated with raw data of the current session. According to some embodiments, the signal associated with a wellness state of the user may include one or more physiological measurements. According to some embodiments, the signals associated with the wellness state of the user include physiological measurements from which the wellness state of the user can be deduced. According to some embodiments, the physiological measurements may include any one or more of breathing rate, heart rate, movement, movement speed and/or frequency, sweat level of the hands of the user, and the like.

According to some embodiments, the raw data may include environmental factors of the surrounding which can affect physiological measurements of the user. According to some embodiments, the raw data may include data associated with a temperature level and/or humidity level surrounding the user. According to some embodiments, the raw data may include data associated with the time of day and/or month in which the current session is performed.

According to some embodiments, the raw data may include the user specific data. According to some embodiments, the system 200/100 may be configured to receive input from the subject before, during, and/or after a session. According to some embodiments, the system 200/100 may be configured to receive input from the subject using the user interface module 212/112. According to some embodiments, the input from the subject may include the user specific data. According to some embodiments, the user specific data may include one or more of the age, gender, medical history of the user, and/or user reported data. According to some embodiments, the user specific data may include data that is passively collected from the user. According to some embodiments, the user reported data may include any one or more of their mood, quality of sleep, lifestyle, events, and the like. According to some embodiments, the passively collected data may be collected using any one or more of electronic devices (such as, e.g., a smartphone), wearable devices, health databases, services and/or systems, and the like.

According to some embodiments, data associated with the current session of the subject 320 may be sent from the apparatus 306/206/106 during and/or after a current session of the user. According to some embodiments, the data associated with the current session of the subject 320 may include any one or more of inputted data and data received from the one or more sensors of the apparatus 306/206/106. According to some embodiments, the data associated with the current session of the subject 320 may include data associated with the indicators 208/108 of the apparatus 306/206/106. According to some embodiments, data associated with the indicators 208/108 of the apparatus 306/206/106 may include the operational parameters of the indicators 208/108 during the current session. According to some embodiments, the data associated with the current session of the subject 320 may include the set of instructions of the current session.

According to some embodiments, data associated with the current session of the subject 320 may be stored within the memory module 304/204. According to some embodiments, the memory module 304/204 may be coupled to and/or integral with the processor. According to some embodiments, the memory module 304/204 may be a cloud storage unit.

According to some embodiments, data associated with the current session of the subject 320 may be sent to an algorithm configured to analyze the data associated with the current session of the subject 320, such as session analysis algorithm 322. According to some embodiments, the program code may include the session analysis algorithm 322. According to some embodiments, the session analysis algorithm 322 may be configured to receive the data associated with the current session. According to some embodiments, the data associated with the current session may be applied to the session analysis algorithm 322 after the current session is completed. According to some embodiments, the data associated with the current session may be applied to the session analysis algorithm 322 during the current session.

According to some embodiments, the session analysis algorithm 322 may be configured to process the data associated with the current session of the user 320. According to some embodiments, the processing may include any one or more of data editing techniques, data reduction techniques, data mining techniques, and the like. According to some embodiments, the session analysis algorithm 322 may be configured to normalize the data associated with the current session of the user 320. According to some embodiments, the session analysis algorithm 322 may be configured to identify the user.

According to some embodiments, the session analysis algorithm 322 may be configured to analyze and/or modify the one or more signals of a sensor of the apparatus 306/206/106 using one or more signals of another sensor of the apparatus 306/206/106. According to some embodiments, the session analysis algorithm 322 may be configured to analyze and/or modify the one or more signals of a sensor of the apparatus 306/206/106 using one or more signals of an accelerometer sensor of the apparatus 306/206/106. According to some embodiments, the session analysis algorithm 322 may be configured to analyze and/or modify the one or more signals of an ECG sensor of the apparatus 306/206/106 using one or more signals of an accelerometer sensor of the apparatus 306/206/106 thereby eliminating noise of the ECG signals.

Advantageously, analyzing and/or modifying the one or more signals of a sensor of the apparatus 306/206/106 using one or more signals of another sensor of the apparats enables the session analysis algorithm 322 to eliminate noise from one or more signals of one or more sensors of the apparatus.

Advantageously, analyzing and/or modifying the one or more signals of a sensor of the apparatus 306/206/106 using one or more signals of an accelerometer sensor may enable removing noise which may be generated by movement of the user and/or the apparatus during the session, from the one or more signals of the sensors of the apparatus.

According to some embodiments, the session analysis algorithm 322 may be configured to generate processed data, which may include data associated with the wellness state of the user based, at least in part, on analyzed raw data. According to some embodiments, and as described in greater detail elsewhere herein, the processed data may include data associated with the wellness state of the user based, at least in part, on data associated with one or more previous sessions of the user. According to some embodiments, and as described in greater detail elsewhere herein, the processed data may include data associated with the wellness state of the user based, at least in part, on one or more comparisons between data associated with one or more previous sessions of the user and data associated with the current session of the user.

According to some embodiments, the session analysis algorithm 322 may be configured to send and/or store the data associated with the current session of the user (the raw and/or the processed data) into a user database 324. According to some embodiments, the user database 324 may be stored within the storage module 304/204. According to some embodiments, the user database 324 may have stored thereon individual user's data, which may be categorized by an identifier of the individual user. According to some embodiments, the identifier of the individual user may include a name, username, and/or ID number or code of the user. According to some embodiments, the session analysis algorithm 322 may be configured to store the data associated with the current session of the user in the user database 324 and/or the storage module 304/204 by identifying the user as being associated with his/her identifier and storing the data of the user with a tag of the identifier of the user. According to some embodiments, the user database 324 may have stored thereon data associated with one or more previous sessions of the user.

According to some embodiments, the session analysis algorithm 322 may be configured to identify the user using the received data. According to some embodiments, the session analysis algorithm 322 may be configured to identify the user using data associated with the current session of the user. According to some embodiments, the session analysis algorithm 322 may be configured to identify the user in real time.

Advantageously, identifying the user may enable the apparatus to be used by more than one user without mixing data of the two users. Moreover, identifying the user may enable the personalization of the configurations and/or the sets of instructions during the sessions.

It is to be understood that once data associated with a current session is stored onto the user database 324 and/or the storage module 304/204, it becomes “data associated with a previous session of the user”. According to some embodiments, once the current session of the user has ended (or has been completed), the data associated with the current session becomes associated with a previous session of the user, or in other words, once a current session is ended it then becomes a previous session. Furthermore, according to some embodiments, the data associated with the current session will be used as “data associated with a previous session of the user” in a future session of the user.

According to some embodiments, the session analysis algorithm 322 may include an algorithm configured to generate a set of instructions for the current session of the user. According to some embodiments, the set of instructions may include operational parameters for the two or more modes of operation of the one or more indicators 208/108 of the apparatus 306/206/106. According to some embodiments, the operational parameters may include data for how to operate the one or more indicators 208/108 in response to signals received from (and/or detected by) the one or more sensors. According to some embodiments, the operational parameters may include data for how to operate the one or more indicators 208/108 in real time in response to signals detected by the one or more sensors. According to some embodiments, the set of instructions may include optional operational parameters for the two or more modes of operation of the one or more indicators 208/108, which may be implemented based on (or in correlation to) the signals received and/or detected during the current session. According to some embodiments, the two or more modes of operation may be configured to sync with signals associated with a current session of the user. According to some embodiments, the two or more modes of operation are configured to sync to a rate being lower than a breathing rate of the user by about 5-30%. According to some embodiments, the two or more modes of operation are configured to sync to a rate being lower than a heart rate of the user by about 5-30%. According to some embodiments, the two or more modes of operation are configured to sync to a rate being lower than a movement rate of the user by about 5-30%.

According to some embodiments, the set of instructions is based, at least in part, on output of the adaptive algorithm 310/210 stored within the storage module 304/204. According to some embodiments, the session analysis algorithm 322 may include the adaptive algorithm 310/210.

According to some embodiments, the adaptive algorithm 310/210 and/or the session analysis algorithm 322 may be configured to receive data associated with one or more previous session of the user 326. According to some embodiments, the data associated with one or more previous session of the user 326 may include data stored within the user database 324. According to some embodiments, the data associated with one or more previous session of the user 326 may include raw data and/or processed data associated with one or more previous session of the user. According to some embodiments, the one or more previous sessions may include a plurality of previous sessions. According to some embodiments, the data associated with one or more previous session may include data from a plurality of previous sessions.

According to some embodiments, the adaptive algorithm 310/210 and/or the session analysis algorithm 322 may be configured to apply the received data (or in other words, the data received by the adaptive algorithm 310/210 and/or the session analysis algorithm 322) to a plurality of configurations 328. According to some embodiments, each of the plurality of configurations 328 may depict one or more optional sets of instructions. According to some embodiments, at least a portion of the plurality of configurations 328 may depict one or more optional sets of instructions. According to some embodiments, the one or more optional sets of instructions may be alternative sets of instructions. According to some embodiments, two or more optional sets of instructions of two or more configurations may be different from one other.

According to some embodiments, the storage module 304/204 may have stored thereon one or more template configurations. According to some embodiments, and as described in greater detail elsewhere herein, the one or more template configurations may be a starting point for the configurations of a specific user, which may then be adjusted over time based on stored/received data associated with sessions of the user.

According to some embodiments, at least a portion of the plurality of configurations 328 may be template configurations. According to some embodiments, at least a portion of the plurality of configurations 328 may be based on one or more template configurations. According to some embodiments, the one or more template configurations may be changed (or adjusted) for an individual user. According to some embodiments, the user database 324 may include data associated with the configurations of the specific/individual user. According to some embodiments, the adaptive algorithm 310/210 and/or the session analysis algorithm 322 may be configured to implement one or more template configurations as one or more of the plurality of configurations. According to some embodiments, the adaptive algorithm 310/210 and/or the session analysis algorithm 322 may be configured to change at least one of the one or more template configurations based, at least in part, on data of the one or more previous sessions of the user. According to some embodiments, the plurality of configurations 328 may be updated periodically from the storage module 304/204.

According to some embodiments, the adaptive algorithm 310/210 and/or the session analysis algorithm 322 may be configured to score each of the plurality of configurations, thereby obtaining a plurality of scores 330. According to some embodiments, each of the scores 330 may be a value associated with one or more key performance indicator(s) (KPI) of the user. According to some embodiments, the adaptive algorithm 310/210 and/or the session analysis algorithm 322 may be configured to score each of the plurality of configurations, wherein the score 330 is based, at least in part, on the combination (or injection of) each of the plurality of configurations with the received data. According to some embodiments, the score and/or the one or more key performance indicator(s) (KPI) of the user may include one or more measures of the performance of the received data in combination with a configuration.

According to some embodiments, the adaptive algorithm 310/210 and/or the session analysis algorithm 322 may be configured to generate a scoring scale configured to score the KPI of at least one of the plurality of configurations with the received data based, at least in part, on data of the at least one previous session of the user. According to some embodiments, the score may be based, at least in part, on the values of the one or more KPI(s) of the user. According to some embodiments, a higher value of the score may be indicative of a better KPI. According to some embodiments, the score and/or the KPI is configured to take into account the needs of the user, such that a higher score and/or higher KPI associated with a specific configuration is correlated with a higher compatibility of the user's needs with the specific configuration.

According to some embodiments, the adaptive algorithm 310/210 and/or the session analysis algorithm 322 may be configured to generate a typecast of the user. According to some embodiments, the scoring scale may be calculated using the typecast of the user. According to some embodiments, the typecast may include one or more identifying features of the user. According to some embodiments, the adaptive algorithm 310/210 and/or the session analysis algorithm 322 may be configured to generate a typecast of the user based on signals associated with the current session.

According to some embodiments, the adaptive algorithm 310/210 and/or the session analysis algorithm 322 may be configured to identify at least one of the plurality of configurations (referred to herein as the one or more identified configurations) as having a preferred score (and/or KPI value). According to some embodiments, the preferred score (and/or KPI value) may be the highest score or relatively highest score, in relation to other scores of other configurations.

According to some embodiments, the adaptive algorithm 310/210 and/or the session analysis algorithm 322 may be configured to generate the set of instructions for the current session, based, at least in part, on the at least one of the plurality of configurations. According to some embodiments, the adaptive algorithm 310/210 and/or the session analysis algorithm 322 may be configured to generate the set of instructions for the current session, based, at least in part, on the identified one or more configurations.

According to some embodiments, the adaptive algorithm 310/210 and/or the session analysis algorithm 322 may be configured to generate the set of instructions for the current session, based, at least in part, on a subset of the plurality of configuration, wherein the scores of the subset of the plurality of configurations are associated with desired KPI values. According to some embodiments, the adaptive algorithm 310/210 and/or the session analysis algorithm 322 may be configured to generate the set of instructions for the current session, based, at least in part, on a subset of the plurality of configuration, wherein the scores of the subset of the plurality of configurations may be the preferred scores, or in other words, based, at least in part, on the one or more identified configurations.

According to some embodiments, the adaptive algorithm 310/210 and/or the session analysis algorithm 322 may be configured to generate the set of instructions by converting the one or more identified configurations into the set of instructions. According to some embodiments, the adaptive algorithm 310/210 and/or the session analysis algorithm 322 may be configured to generate the set of instructions by converting an identified configuration into the set of instructions. According to some embodiments, the adaptive algorithm 310/210 and/or the session analysis algorithm 322 may be configured to generate the set of instructions by combining two or more identified configurations and converting the combination thereof into the set of instructions.

According to some embodiments, the adaptive algorithm 310/210 and/or the session analysis algorithm 322 may be configured to generate the set of instructions for the current session, based, at least in part, on at least two configurations, wherein each of the at least two configurations is associated with data of different previous sessions of the user.

According to some embodiments, the adaptive algorithm 310/210 and/or the session analysis algorithm 322 may be configured to generate the set of instructions for the current session, based, at least in part, on any one or more of the age, gender, environmental factors of the current session and/or geographical region, user inputted data, raw data of the user and/or the current session, processed data associated with the user and/or with the current session, medical history of the user, user reported data, and data that is passively collected from the user. According to some embodiments, the adaptive algorithm 310/210 and/or the session analysis algorithm 322 may be configured to generate the set of instructions for the current session, based, at least in part, on information received from health databases, services and/or systems, which may be sent directly to the processor 202/102 and/or storage module 304/204 or inputted by the user.

According to some embodiments, the adaptive algorithm 310/210 and/or the session analysis algorithm 322 may be configured to adjust the one or more configurations. According to some embodiments, the adaptive algorithm 310/210 and/or the session analysis algorithm 322 may be configured to adjust the one or more configurations based on the scores and/or KPI(s) of the configurations. In other words, according to some embodiments, the configurations may change over time for a specific user and may change between sessions of a user. According to some embodiments, the configurations may become more personalized for a specific user over time. According to some embodiments, the configurations may start (i.e., at a first session of the user) as the template configurations and over time get adjusted into more personalized configurations for the specific user.

According to some embodiments, the configurations of the user may be stored and/or periodically updated at the storage module 304/204. According to some embodiments, the adaptive algorithm 310/210 and/or the session analysis algorithm 322 may be configured to store and/or record any one or more of the data associated with the user, the data including any one or more of signals obtained during the session, one or more configurations having one or more scores indicating the KPI of the user, environmental data, data inputted by the user, and/or the set of instructions of the session.

According to some embodiments, the adaptive algorithm 310/210 and/or the session analysis algorithm 322 may be configured to generate the set of instructions based, at least in part, on the current session of the user. According to some embodiments, the adaptive algorithm 310/210 and/or the session analysis algorithm 322 may be configured to generate the set of instructions in real time. In other words, according to some embodiments, the adaptive algorithm 310/210 and/or the session analysis algorithm 322 may be configured to continuously and/or periodically generate new sets of instructions for the user during the user's current session.

According to some embodiments, the adaptive algorithm 310/210 and/or the session analysis algorithm 322 may be configured to receive raw and/or processed data associated with the current session in real time. According to some embodiments, the adaptive algorithm 310/210 and/or the session analysis algorithm 322 may be configured to receive data associated with one or more signals of the current session in real time. According to some embodiments, the adaptive algorithm 310/210 and/or the session analysis algorithm 322 may be configured to receive data associated with the current session of the subject 320 in real time. According to some embodiments, the adaptive algorithm 310/210 and/or the session analysis algorithm 322 may be configured to process the raw data associated with the current session of the subject in real time. According to some embodiments, the received data, as described hereinabove, may include the data associated with the current session of the subject 320 received in real time.

According to some embodiments, the adaptive algorithm 310/210 and/or the session analysis algorithm 322 may be configured to apply the received data to a plurality of configurations.

According to some embodiments, the adaptive algorithm 310/210 and/or the session analysis algorithm 322 may be configured to apply the received data to the plurality of configurations 328. According to some embodiments, each of the plurality of configurations 328 may depict one or more optional sets of instructions, as described in greater detail hereinabove. According to some embodiments, the adaptive algorithm 310/210 and/or the session analysis algorithm 322 may be configured to score each of the plurality of configurations, thereby obtaining a plurality of scores, each of the scores being values associated with key performance indicator(s) (KPI) of the user. According to some embodiments, the adaptive algorithm 310/210 and/or the session analysis algorithm 322 may be configured to identify at least one of the plurality of configurations as having a preferred score (and/or KPI value). According to some embodiments, the adaptive algorithm 310/210 and/or the session analysis algorithm 322 may be configured to generate the set of instructions for the current session, based, at least in part, on the at least one of the plurality of configurations. According to some embodiments, the adaptive algorithm 310/210 and/or the session analysis algorithm 322 may be configured to generate the set of instructions for the current session in real time, based, at least in part, on data associated with previous sessions of the user.

### Method for Inducing Wellness in a User

According to some embodiments, there is provided a method for inducing wellness in a user. According to some embodiments, the method may include receiving at least one signal associated with a current session of a user, from at least one sensor positioned on an apparatus configured to be proximal to the user. According to some embodiments, the method may include applying to the apparatus, a set of instructions for a current session, wherein the set of instructions includes operational parameters for two or more modes of operation of an indicator of the apparatus. According to some embodiments, the method may include generating the set of instructions. According to some embodiments, the method may include generating the set of instructions based, at least in part, on output of an algorithm, such as the adaptive algorithm 310/210 and/or the session analysis algorithm 322 as described hereinabove.

According to some embodiments, the method may include a biofeedback method of inducing wellness in a user. According to some embodiments, the method may include positioning the apparatus 306/206/106 in proximity to a user. According to some embodiments, the method may include positioning the apparatus the apparatus 306/206/106 in contact with the user. According to some embodiments, the method may include holding the apparatus 306/206/106. According to some embodiments, the method may include receiving at least two indications from the one or more indicators 208/108 of the apparatus 306/206/106, thereby self-adjusting the wellness level of the user. According to some embodiments, the method may include operating the one or more indicators 208/108 of the apparatus 306/206/106, according to the set of instructions of the current session.

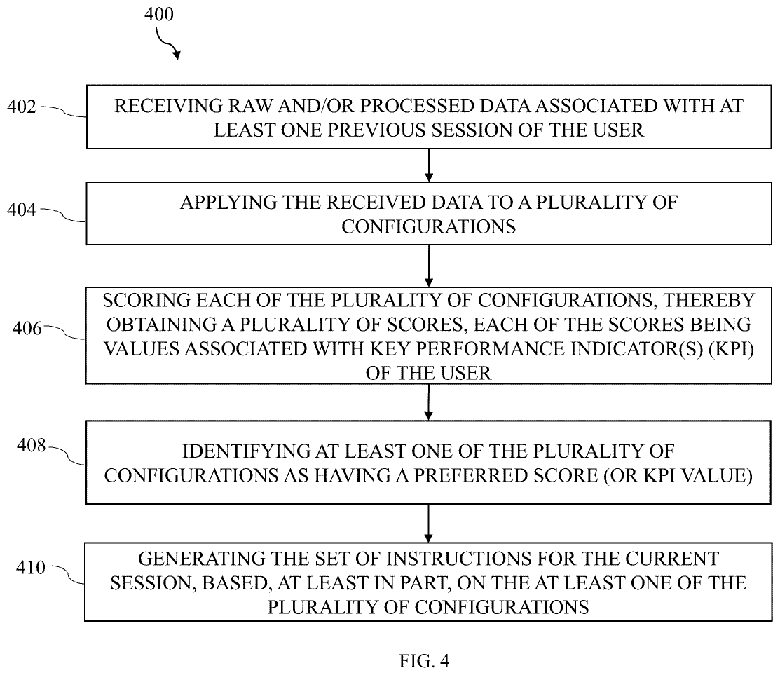

Reference is made to FIG. 4, which shows a flow diagram of steps of a method for generating a set of instructions, in accordance with some embodiments of the present invention.

According to some embodiments, at step 402, the method 400 for generating a set of instructions, may include receiving raw and/or processed data associated with at least one previous session. According to some embodiments, at step 404, the method 400 may include applying the received data to a plurality of configurations. According to some embodiments, at step 406, the method 400 may include scoring each of the plurality of configurations, thereby obtaining a plurality of scores, each of the scores being values associated with key performance indicator(s) (KPI) of the user. According to some embodiments, at step 408, the method may include identifying at least one of the plurality of configurations as having a preferred score (or KPI value). According to some embodiments, at step 410, the method may include generating the set of instructions for the current session, based, at least in part, on the at least one of the plurality of configurations.

According to some embodiments, the method 400 may include performing the current session. According to some embodiments, the method 400 may include generating the set of instructions of the current session using the processor 202/102. According to some embodiments, the method 400 may include generating and/or receiving the set of instructions of the current session by the processor 202/102 during the current session (or in other words, in real time).

According to some embodiments, the method 400 may include monitoring and/or recording the session of the user/subject. According to some embodiments, the method 400 may include monitoring and/or recording the session of the subject by monitoring and/or recording the signals of the one or more sensors of the apparatus 306/206/106. According to some embodiments, the method 400 may include monitoring and/or recording the session of the subject by monitoring and/or recording data received from the one or more sensors of the apparatus 306/206/106.

According to some embodiments, the signals, or in other words, the signals associated with the current session, may include a signal associated with a wellness state of the user and/or a signal associated with raw data of the current session. According to some embodiments, the signal associated with a wellness state of the user may include one or more physiological measurements. According to some embodiments, the signals associated with the wellness state of the user include physiological measurements from which the wellness state of the user can be deduced. According to some embodiments, the physiological measurements may include any one or more of breathing rate, heart rate, movement, movement speed and/or frequency, sweat level of the hands of the user, and the like.

According to some embodiments, the raw data may include environmental factors of the surrounding which can affect physiological measurements of the user. According to some embodiments, the raw data may include data associated with a temperature level and/or humidity level surrounding the user. According to some embodiments, the raw data may include data associated with the time of day and/or month in which the current session is performed.

According to some embodiments, the raw data may include the user specific data. According to some embodiments, the method may include receiving input from the subject before, during, and/or after a session. According to some embodiments, the method may include receiving input from the subject using the user interface module 212/112. According to some embodiments, the input from the subject may include the user specific data. According to some embodiments, the user specific data may include one or more of the age, gender, medical history of the user, and/or user reported data. According to some embodiments, the user specific data may include data that is passively collected from the user. According to some embodiments, the method may include passively collecting data from the user

According to some embodiments, the data associated with the current session of the subject 320 may include any one or more of inputted data and data received from the one or more sensors of the apparatus 306/206/106. According to some embodiments, the data associated with the current session of the subject 320 may include data associated with the indicators 208/108 of the apparatus 306/206/106. According to some embodiments, data associated with the indicators 208/108 of the apparatus 306/206/106 may include the operational parameters of the indicators 208/108 during the current session. According to some embodiments, the data associated with the current session of the subject 320 may include the set of instructions of the current session.

According to some embodiments, the method may include storing the data associated with the current session of the subject 320 within the memory module 304/204. According to some embodiments, the method may include sending the data associated with the current session of the subject 320 to an algorithm configured to analyze the data associated with the current session of the subject 320, such as session analysis algorithm 322.

According to some embodiments, the method may include applying the data associated with the current session to the session analysis algorithm 322 after the current session is completed. According to some embodiments, the method may include applying the data associated with the current session to the session analysis algorithm 322 during the current session.

According to some embodiments, the method may include processing the data associated with the current session of the user 320, thereby generating processed data. According to some embodiments, the processing may include any one or more of data editing techniques, data reduction techniques, data mining techniques, and the like. According to some embodiments, the method may include normalizing the data associated with the current session of the user 320. According to some embodiments, method may include identifying the user. According to some embodiments, method may include identifying the user based, at least in part, on the received data.

According to some embodiments, the method may include analyzing and/or modifying the one or more signals of a sensor of the apparatus 306/206/106 using one or more signals of another sensor of the apparatus 306/206/106. According to some embodiments, the method may include analyzing and/or modifying the one or more signals of a sensor of the apparatus 306/206/106 using one or more signals of an accelerometer sensor of the apparatus 306/206/106. According to some embodiments, the method may include analyzing and/or modifying the one or more signals of an ECG sensor of the apparatus 306/206/106 using one or more signals of an accelerometer sensor of the apparatus 306/206/106, thereby eliminating noise of the ECG signals.

Advantageously, analyzing and/or modifying the one or more signals of a sensor of the apparatus 306/206/106 using one or more signals of another sensor of the apparats enables the session analysis algorithm 322 to eliminate noise from one or more signals of one or more sensors of the apparatus.

Advantageously, analyzing and/or modifying the one or more signals of a sensor of the apparatus 306/206/106 using one or more signals of an accelerometer sensor may enable removing noise which may be generated by movement of the user and/or the apparatus during the session, from the one or more signals of the sensors of the apparatus.

According to some embodiments, the method may include generating processed data, which may include data associated with the wellness state of the user based, at least in part, on analyzed raw data. According to some embodiments, and as described in greater detail elsewhere herein, the processed data may include data associated with the wellness state of the user based, at least in part, on data associated with one or more previous sessions of the user. According to some embodiments, and as described in greater detail elsewhere herein, the processed data may include data associated with the wellness state of the user based, at least in part, on one or more comparisons between data associated with one or more previous sessions of the user and data associated with the current session of the user.

According to some embodiments, the method may include sending and/or storing the data associated with the current session of the user (the raw and/or the processed data) into the user database 324. According to some embodiments, the method may include storing the data associated with the current session of the user in the user database 324 and/or the storage module 304/204 by identifying the user as being associated with his/her identifier and storing the data of the user with the identifier of the user.

According to some embodiments, the method may include receiving data associated with one or more previous session of the user 326. According to some embodiments, the data associated with one or more previous session of the user 326 may include data stored within the user database 324. According to some embodiments, the data associated with one or more previous session of the user 326 may include raw data and/or processed data associated with one or more previous session of the user. According to some embodiments, the one or more previous sessions may include a plurality of previous sessions. According to some embodiments, the data associated with one or more previous session may include data from a plurality of previous sessions.

According to some embodiments, the method may include applying the received data to a plurality of configurations 328. According to some embodiments, each of the plurality of configurations 328 may depict one or more optional sets of instructions. According to some embodiments, at least a portion of the plurality of configurations 328 may depict one or more optional sets of instructions. According to some embodiments, the one or more optional sets of instructions may be alternative sets of instructions. According to some embodiments, two or more optional sets of instructions of two or more configurations may be different from one other.

According to some embodiments, at least a portion of the plurality of configurations 328 may be template configurations. According to some embodiments, at least a portion of the plurality of configurations 328 may be based on one or more template configurations. According to some embodiments, the one or more template configurations may be changed (or adjusted) for an individual user. According to some embodiments, the user database 324 may include data associated with the configurations of the specific/individual user.

According to some embodiments, the method may include implementing one or more template configurations as one or more of the plurality of configurations. According to some embodiments, the method may include changing (or adjusting) at least one of the one or more template configurations based, at least in part, on data of the one or more previous sessions of the user. According to some embodiments, the method may include updating the plurality of configurations 328 periodically in the storage module 304/204.

According to some embodiments, the method may include scoring each of the plurality of configurations, thereby obtaining a plurality of scores 330. According to some embodiments, each of the scores 330 may be a value associated with one or more key performance indicator(s) (KPI) of the user. According to some embodiments, the method may include scoring each of the plurality of configurations, wherein the score 330 is based, at least in part, on the combination (or injection of) each of the plurality of configurations with the received data. According to some embodiments, the score and/or the one or more key performance indicator(s) (KPI) of the user may include one or more measures of the performance of the received data in combination with a configuration.

According to some embodiments, the method may include generating a scoring scale configured to score the KPI of at least one of the plurality of configurations with the received data based, at least in part, on data of the at least one previous session of the user. According to some embodiments, the score may be based, at least in part, on the values of the one or more KPI(s) of the user. According to some embodiments, a higher value of the score may be indicative of a better KPI. According to some embodiments, the score and/or the KPI is configured to take into account the needs of the user, such that a higher score and/or higher KPI associated with a specific configuration is correlated with a higher compatibility of the user's needs with the specific configuration.

According to some embodiments, the method may include generating a typecast of the user. According to some embodiments, the scoring scale may be calculated using the typecast of the user. According to some embodiments, the typecast may include one or more identifying features of the user. According to some embodiments, the method may include generating a typecast of the user based on signals associated with the current session.

According to some embodiments, the method may include identifying at least one of the plurality of configurations (referred to herein as the one or more identified configurations) as having a preferred score (and/or KPI value). According to some embodiments, the preferred score (and/or KPI value) may be the highest score or relatively highest score, in relation to other scores of other configurations.

According to some embodiments, the method may include generating the set of instructions for the current session, based, at least in part, on the at least one of the plurality of configurations. According to some embodiments, the method may include generating the set of instructions for the current session, based, at least in part, on the identified one or more configurations.

According to some embodiments, the method may include generating the set of instructions for the current session, based, at least in part, on a subset of the plurality of configuration, wherein the scores of the subset of the plurality of configurations are associated with desired KPI values. According to some embodiments, the method may include generating the set of instructions for the current session, based, at least in part, on a subset of the plurality of configuration, wherein the scores of the subset of the plurality of configurations may be the preferred scores, or in other words, based, at least in part, on the one or more identified configurations.

According to some embodiments, the method may include generating the set of instructions by converting the one or more identified configurations into the set of instructions. According to some embodiments, the method may include generating the set of instructions by converting an identified configuration into the set of instructions. According to some embodiments, the method may include generating the set of instructions by combining two or more identified configurations and converting the combination thereof into the set of instructions.

According to some embodiments, the method may include generating the set of instructions for the current session, based, at least in part, on at least two configurations, wherein each of the at least two configurations is associated with data of different previous sessions of the user.

According to some embodiments, the method may include generating the set of instructions for the current session, based, at least in part, on any one or more of the age, gender, environmental factors of the current session and/or geographical region, user inputted data, raw data of the user and/or the current session, processed data associated with the user and/or with the current session, medical history of the user, user reported data, and data that is passively collected from the user. According to some embodiments, the method may include generating the set of instructions for the current session, based, at least in part, on information received from health databases, services and/or systems, which may be sent directly to the processor 202/102 and/or storage module 304/204 or inputted by the user.

According to some embodiments, the method may include adjusting (or in other words, making changes to) the one or more configurations. According to some embodiments, the method may include adjusting the one or more configurations based on the scores and/or KPI(s) of the configurations. According to some embodiments, the configurations may change over time for a specific user and may change between sessions of a user. According to some embodiments, the method may include personalizing the configurations over time. According to some embodiments, the configurations may start (i.e., at a first session of the user) as the template configurations and over time get adjusted into more personalized configurations for the specific user.

According to some embodiments, the method may include storing and/or updating the configurations of the user periodically at the storage module 304/204. According to some embodiments, the method may include storing and/or recording any one or more of the data associated with the user, the data including any one or more of signals obtained during the session, one or more configurations having one or more scores indicating the KPI of the user, environmental data, data inputted by the user, and/or the set of instructions of the session.

According to some embodiments, the method may include generating the set of instructions based, at least in part, on the current session of the user. According to some embodiments, the method may include generating the set of instructions in real time. According to some embodiments, the method may include continuously and/or periodically generating new sets of instructions for the user during the user's current session.

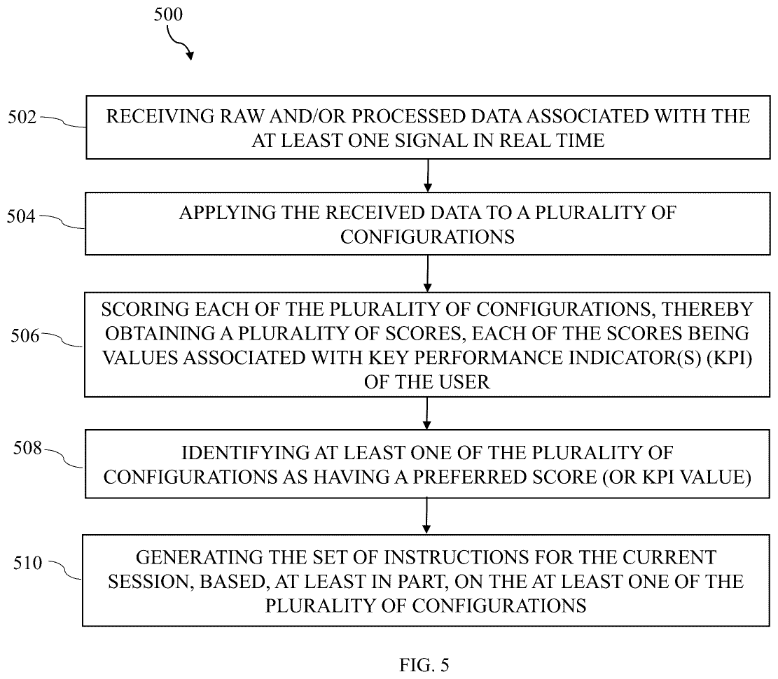

Reference is made to FIG. 5, which shows a flow diagram of steps of a method for generating a set of instructions in real time, in accordance with some embodiments of the present invention. According to some embodiments, the method 500 may include all or some of the steps of method 400. According to some embodiments, the method 400 may include all or some of the steps of method 500.

According to some embodiments, at step 502, the method 500/400 may include receive raw and/or processed data associated with the at least one signal in real time. According to some embodiments, at step 504, the method 500/400 may include applying the received data to a plurality of configurations. According to some embodiments, at step 506, the method 500/400 may include scoring each of the plurality of configurations, thereby obtaining a plurality of scores, each of the scores being values associated with key performance indicator(s) (KPI) of the user. According to some embodiments, at step 508, the method 500/400 may include identifying at least one of the plurality of configurations as having a preferred score (or KPI value). According to some embodiments, at step 510, the method 500/400 may include generate the set of instructions for the current session, based, at least in part, on the at least one of the plurality of configurations.

According to some embodiments, the method 500/400 may include receiving raw and/or processed data associated with the current session, in real time. According to some embodiments, the method 500/400 may include receiving data associated with one or more signals of the current session in real time. According to some embodiments, the method 500/400 may include processing the raw data associated with the current session of the subject in real time. According to some embodiments, the received data, as described hereinabove, may include the data associated with the current session of the subject 320 received in real time.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods according to some embodiments may be described in a specific sequence, methods of the disclosure may include some or all of the described stages carried out in a different order. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

1. A wellness inducing system, comprising:
an apparatus configured to be proximal to a user, the apparatus comprising:
at least one sensor configured to collect signals associated with a current session of a user; and
an indicator having at least two modes of operation;
a processor in communication with a memory module, the memory module having stored thereon program code, the program code executable by the processor to:
receive at least one signal from the at least one sensor; and
apply, to the apparatus, a set of instructions for the current session, wherein the set of instructions comprises operational parameters for the at least two modes of operation, and wherein the set of instructions is based, at least in part, on output of an algorithm configured to:
receive raw and/or processed data associated with at least one previous session of the user;
apply the received data to a plurality of configurations;
score each of the plurality of configurations, thereby obtaining a plurality of scores, each of the scores being values associated with key performance indicator(s) (KPI) of the user;
identify at least one of the plurality of configurations as having a preferred score (or KPI value); and
generate the set of instructions for the current session, based, at least in part, on the at least one of the plurality of configurations.

2. The system of claim 1, wherein a higher value of the score is indicative of a better KPI.

3. The system of claim 1, wherein the signals associated with a current session of a user comprise at least one of a signal associated with a wellness state of the user and/or a signal associated with raw data of the current session, wherein signals associated with the wellness state of the user comprise physiological measurements from which the wellness state of the user can be deduced.

4. (canceled)

5. The system of claim 1, wherein the raw data comprises environmental factors of the surrounding which can affect physiological measurements of the user.

6. The system of claim 1, wherein the raw data comprises user specific data, the user specific data comprising any one or more of the age, gender, medical history, user reported data, and data that is passively collected from the user.

7. The system of claim 1, wherein the processed data comprises data associated with the wellness state of the user based, at least in part, on analyzed raw data.

8. The system of claim 1, wherein the set of instructions comprises optional operational parameters for the at least two modes of operation of the indicator, to be implemented based on (or in correlation to) the signals associated with a current session of a user.

9. The system of claim 1, wherein each configuration depicts one or more optional sets of instructions.

10. The system of claim 1, wherein the program code is further executable to identify the user.

11. The system of claim 1, wherein the at least two modes of operation are configured to sync with signals associated with a current session of the user.

12. The system of claim 11, wherein the signals are associated with at least one of a breathing rate, heart rate, and movement of the user.

13. The system of claim 11, wherein the at least two modes of operation are configured to sync to a rate being lower than a breathing rate or heart rate and movement of the user, by about 5-30%.

14. The system of claim 1, wherein the at least one of previous sessions comprises a plurality of previous sessions.

15. The system of claim 1, wherein the program code is further executable to store and/or record data associated with the user, the data comprising any one or more of signals obtained during the session, one or more configurations having one or more scores indicating the KPI of the user, environmental data, data inputted by the user, and the set of instructions of the session.

16. The system of claim 1, wherein the algorithm is further configured to implement one or more template configurations for the plurality of configurations.

17. The system of claim 16, wherein the algorithm is further configured to change at least one of the one or more template configurations based, at least in part, on data of the at least one previous session of the user.

18. The system of claim 1, wherein generating the set of instructions for the current session is based, at least in part, on a subset of the plurality of configuration, wherein the scores of the subset of the plurality of configurations are associated with desired KPI values.

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. A biofeedback method of inducing wellness comprising:
positioning the apparatus of claim 1 in proximity to a user; and
receiving at least two indications from the indicator, thereby self-adjusting the wellness level of the user.

41. The method of claim 40, wherein positioning the apparatus in proximity to the user comprises positioning the apparatus in contact with the user.

42. A method for inducing wellness, comprising;
receiving at least one signal associated with a current session of a user, from at least one sensor positioned on an apparatus configured to be proximal to the user;
applying to the apparatus, a set of instructions for a current session, wherein the set of instructions comprises operational parameters for at least two modes of operation of an indicator of the apparatus;
wherein the set of instructions is based, at least in part, on output of an algorithm configured to:
receive raw and/or processed data associated with at least one previous session;
apply the received data to a plurality of configurations;
score each of the plurality of configurations, thereby obtaining a plurality of scores, each of the scores being values associated with key performance indicator(s) (KPI) of the user;
identify at least one of the plurality of configurations as having a preferred score (or KPI value); and
generate the set of instructions for the current session, based, at least in part, on the at least one of the plurality of configurations.

43. (canceled)

44. (canceled)

45. (canceled)

46. (canceled)

47. (canceled)

48. (canceled)
